(19) United States
(12) Patent Application Publication
(10) Pub. No.: US 2004/0087947 A1
(43) Pub. Date: May 6, 2004

(54) Minimally invasive expanding spacer and method

(76) Inventors: Roy Lim, Cordova, TN (US); Michael C. Sherman, Memphis, TN (US)

Correspondence Address: COATS & BENNETT, PLLC, P O BOX 5, RALEIGH, NC, 27602, US

(21) Appl. No.: 10/229,560

(22) Filed: Aug. 28, 2002

(51) Int. Cl. A61B 17/70

(52) U.S. Cl. 606/61

(57) ABSTRACT

A spacer and method of using the spacer for positioning between and spacing vertebral members. The spacer is positionable between a first position having a reduced size to be minimally invasive when inserted into the patient between the vertebral members. The spacer is expandable up to a second position to contact the vertebral members. In one embodiment, a delivery device is attached to position the spacer within the patient. The delivery device may remain connected to the spacer, or may be removable with the spacer remaining within the patient as the delivery device is removed. In one embodiment, the spacer comprises a first member and a second member each positioned about a middle member. Middle member moves relative to the first and second members with angled surfaces contacting to deploy the spacer between the closed and open orientations.

### BACKGROUND

Various devices are used for controlling the spacing between vertebral members. These devices may be used on a temporary basis, such as during surgery when it is necessary to access the specific surfaces of the vertebral member. One example includes preparing the endplates of a vertebral member. The devices may also remain permanently within the patient to space the vertebral members.

It is often difficult to position the device between the vertebral members in a minimally invasive manner. A device that is small may be inserted into the patient and between the vertebral members in a minimally invasive manner. However, the small size may not be adequate to effectively space the vertebral members. A larger device may be effective to space the vertebral members, but cannot be inserted into the patient and between the vertebral members in a minimally invasive manner.

### SUMMARY

The present invention is directed to a device for spacing vertebral members. The device is positionable between a first orientation having a minimum height, and a second orientation having a maximum height. The device includes a first member having a first angled surface that extends in a first direction, and a second member having a second angled surface that extends in a second direction. As the first member is moved relative to the second member, the angled surfaces contact each other and the size of the spacer increases. The device may be positioned on a delivery device such that it can be removed from the patient when the procedure is completed, or may be detachable from the delivery device to remain within the patient, either permanently or for a predetermined period.

In one embodiment of using the device, the device is positioned between two vertebral members. During the positioning, the device is in a closed orientation having a small size to facilitate insertion and be minimally invasive to the patient. Once positioned, the members are moved relative to each other such that the angled surfaces contact each other. The contact and movement of the members causes the overall height of the spacer to increase. The spacer may be positioned within a variety of heights depending upon the application. In some embodiments, the spacer includes a locking means to locking the spacer at a height and prevent the spacer from closing.

### BRIEF DESCRIPTION OF THE DRAWINGS

FIG. 1 is a perspective view illustrating a spacer in a closed orientation and attached to a delivery device constructed according to one embodiment of the present invention;

FIG. 2 is a perspective view illustrating a spacer in an open orientation with the delivery device removed constructed according to one embodiment of the present invention;

FIG. 3 is a is perspective view illustrating one embodiment of a middle member attached to a delivery device constructed according to one embodiment of the present invention;

FIG. 4 is a cross-sectional view of one embodiment of the middle member constructed according to one embodiment of the present invention;

FIG. 5 is a perspective view illustrating a chassis constructed according to one embodiment of the present invention;

FIG. 6 is a partial perspective view illustrating locking tabs on the chassis mating with indents on the middle member in accordance with one embodiment of the present invention;

FIG. 7 is a side view of the first member constructed according to one embodiment of the present invention;

FIG. 8 is a side view of the second member constructed according to one embodiment of the present invention;

FIG. 9 is a cross-section view of a first member, second member, and middle member in a closed orientation according to one embodiment of the present invention;

FIG. 10 is a cross-section view of a first member, second member, and middle member in a partially deployed orientation according to one embodiment of the present invention;

FIG. 11 is a cross-section view of a first member, second member, and middle member in a fully deployed orientation according to one embodiment of the present invention;

FIG. 12 is a partial perspective view of an alternative embodiment of a middle member;

FIG. 13 is a perspective view of an alternative embodiment of a chassis;

FIG. 14 is a perspective view of a locking member constructed according to one embodiment of the present invention; and

FIG. 15 is a perspective view of a spacer constructed according to one embodiment of the present invention.

### DETAILED DESCRIPTION

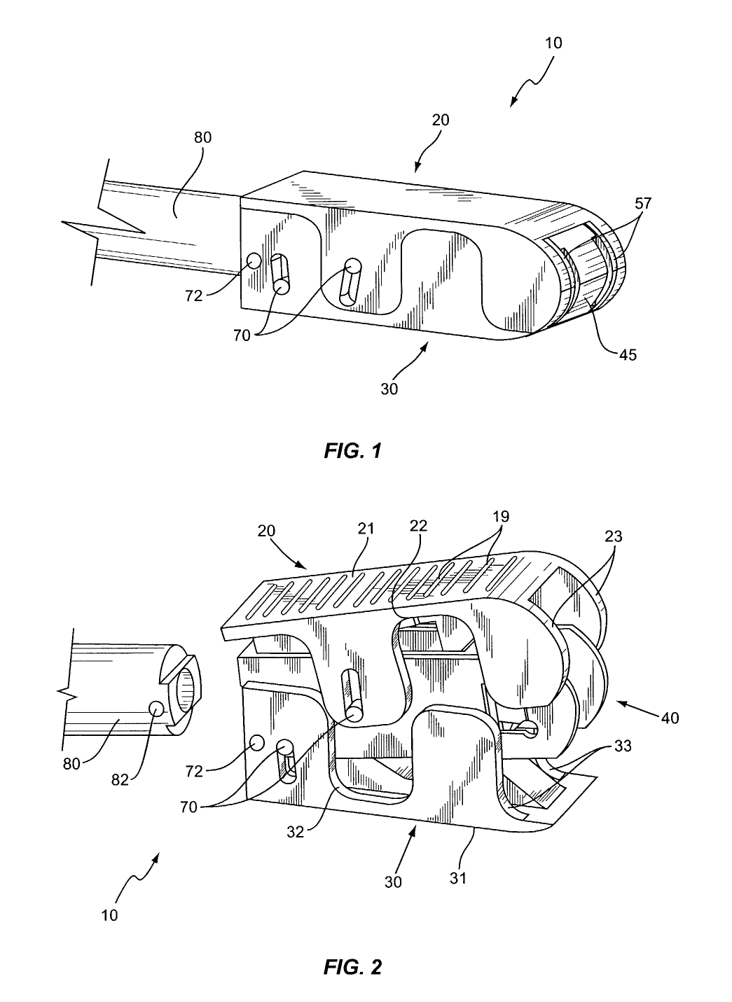

The present invention is directed to a spacer, generally indicated as 10, for positioning between vertebral members. The spacer 10 is adjustable between a first position as illustrated in FIG. 1 having a reduced size to be minimally invasive when inserted into the patient between the vertebral members. The spacer 10 is expandable up to a second position as illustrated in FIG. 2 to contact the vertebral members. The spacer 10 may be expandable to a variety of different heights depending upon the desired application. In one embodiment, a delivery device 80 is attached to position the spacer 10 within the patient. The delivery device 80 may remain connected to the spacer 10, or may be removable with the spacer 10 remaining within the patient as the delivery device 80 is removed. In one embodiment, spacer 10 comprises a first member 20 and a second member 30 each positioned about a middle member 40. Middle member 40 moves relative to the first and second members 20, 30 to deploy the spacer 10 between the closed and open orientations.

In one embodiment, both first member 20 and second member 30 have a substantially U-shaped configuration having respectively a contact surface 21 for contacting a first vertebral member, and a contact surface 31 for contacting a second vertebral member. Sidewalls 23, 33 extend respectively from the contact surfaces 21, 31. In one embodiment as illustrated in FIG. 1, sidewalls 23, 33 have complimentary shapes to mate together in the closed orientation. Edges 22 of first member 20 and edges 32 of second member 30 are adjacently positioned in the closed orientation to reduce the overall size of the spacer 10. In one embodiment as illustrated in FIGS. 1 and 2, sidewalls 23, 33 have complimentary curved edges 22, 32 that extend differing amounts from the contact surfaces 21, 31. In another embodiment, sidewalls 23, 33 are substantially straight and positioned a constant amount from the contact surfaces 21, 31. Sidewalls 23, 33 may have a variety of different shapes and are considered to be included within the scope of the present invention. Edges 22, 32 may contact one another when the spacer 10 is in the closed orientation, or may be spaced apart a distance. In one embodiment, contact surfaces 21, 31 are substantially flat as illustrated in FIG. 1. In another embodiment, contact surfaces 21, 31 have stabilization features 19 such as ridges or knurled surfaces to contact the vertebral members.

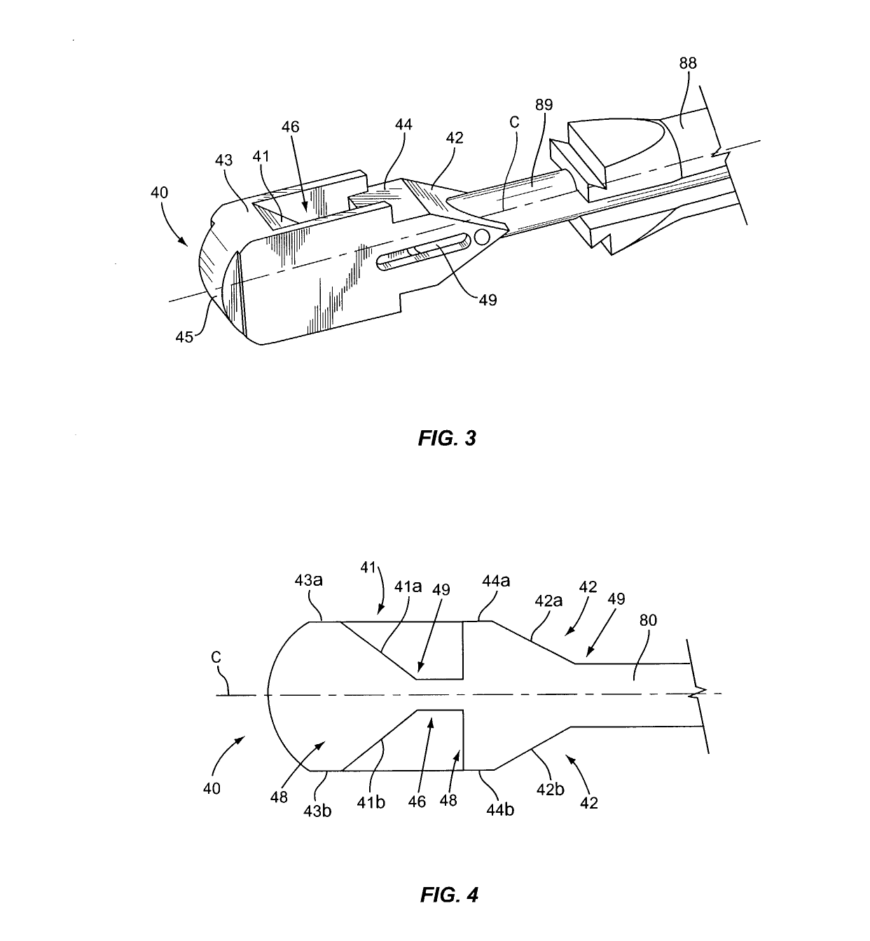

The middle member 40 moves relative to the first and second members 20, 30 to deploy the spacer 10 from the closed orientation to the open orientation. FIG. 3 illustrates one embodiment of the middle member 40 and includes a first wedge 41 and a second wedge 42, and FIG. 4 illustrates a cross-sectional view of the middle member 40. First wedge 41 includes first and second angled surfaces 41a, 41b and second wedge 42 comprises first and second angled surfaces 42a, 42b. The angled surfaces converge towards a vertex end 49 of the wedges and diverge to a base end 48. The wedges 41, 42 are each positioned with the vertex end 49 positioned proximal to the base end 48 (i.e., the vertex end 49 is positioned closer to a proximal end of the spacer 10 than the base end 48). Wedges 41, 42 may be positioned at a variety of locations along the spacer 10. In one embodiment, wedges 41, 42 are separated with a space 46 positioned between the vertex end 49 of the first wedge 41 and the base end 48 of the second wedge 42.

Wedges 41, 42 may be positioned at a variety of angles and sizes. In one embodiment as illustrated in FIGS. 3 and 4, the angled surfaces of the two wedges 41, 42 are different. By way of example, the angled surfaces 41a, 41b of wedge 41 have a greater slope than the angled surfaces 42a, 42b of wedge 42. In another embodiment, a first side of the middle member 40 has different slope than a second side of the middle member 40. By way of example, angled surface 41a may have a different slope than angled surface 41b, and angled surface 42a may have a different slope than angled surface 42b. In another embodiment, both wedges 41, 42 have angled surfaces with the same slope. Various combinations of slope differences may be included within the present invention. In one embodiment, the angles of angled edges 41a, 41b, 42a, and 42b may range from about 20° to about 40°. In one embodiment, the wedges 41, 42 have different lengths. By way of example, wedge 41 has a length that is longer than wedge 42. In one embodiment, a first side of a wedge may have a longer length than a second side of a wedge. For example, angled surface 41a may be longer than angled surface 41b.

In one embodiment, a platform surface 43, 44 is positioned at the base end 48 of each angled surface of wedges 41, 42. As illustrated in FIG. 4, wedge 41 comprises platform surfaces 43a, 43b, and wedge 42 comprises 44a, 44b. Platform surfaces 43, 44 provide a positioning surface for the first and second members 20, 30 to contact in when in the open orientation as will be explained in more detail. Platform surfaces 43, 44 are aligned at different angles then the angled surfaces. By way of example using the embodiment illustrated in FIG. 4, angled surfaces 41a, 41b are aligned at a different angle relative to the centerline C of the spacer 10 than platform surfaces 43a, 43b, and angled surfaces 42a, 42b are at different angles than platform surfaces 44a, 44b. In one embodiment, platform surfaces 43, 44 are substantially parallel with the centerline C.

Middle member 40 further comprises a rounded front section 45 shaped to ease the insertion of the spacer 10 into the patient. Front section 45 may extend across the entire width of the middle member 40, or a limited distance of the width.

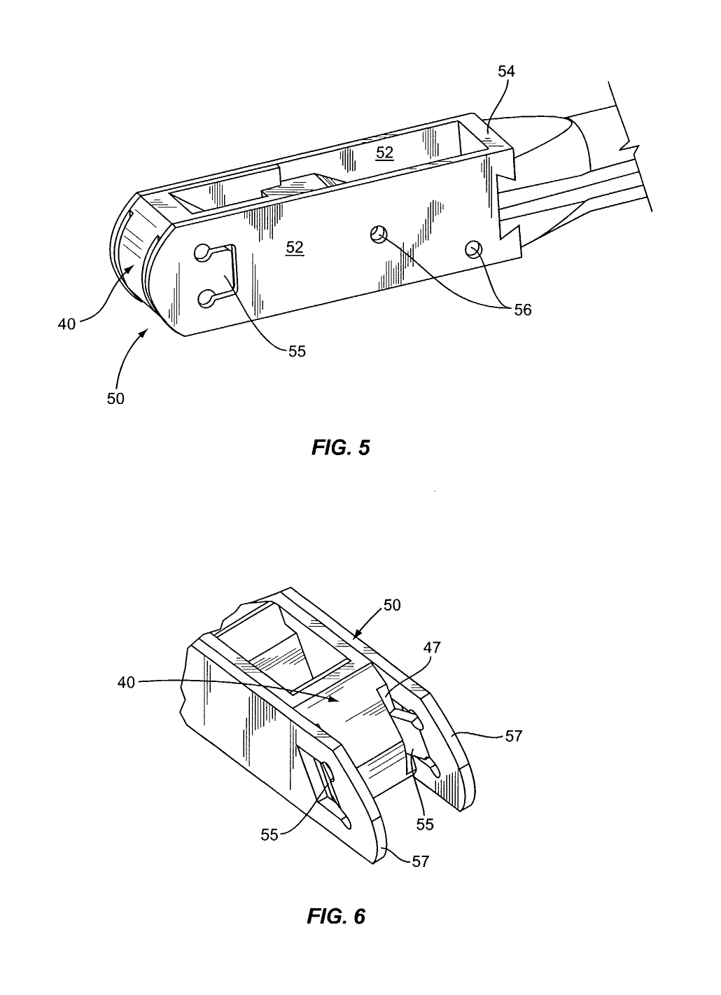

Middle member 40 may further include a chassis 50 as illustrated in FIG. 5. In one embodiment, chassis 50 is substantially U-shaped and includes a pair of sidewalls 52 and a proximal member 54. Proximal member 54 spans between the two sidewalls 52 and includes an opening sized to receive the middle member 40. Sidewalls 52 are spaced apart a distance to extend along a first and second side of the middle member 40. The sidewalls 52 are spaced a distance apart to allow the middle member 40 positioned therebetween to move between a first and second position to move the spacer 10 between the open and closed orientations.

Locking tabs 55 are positioned on the sidewalls 52 to extend outward and mate with the middle member 40. Middle member 40 includes indents 47 positioned along a side edge to receive the locking tabs 55 as illustrated in FIG. 6. The locking tabs 55 flex inward to contact the indents 47 and prevent the middle member 40 from moving in a distal direction. In use, when the spacer 10 is in the closed orientation, the front 45 of the middle member 40 is aligned substantially with the chassis front edges 57 as illustrated in FIG. 1. As the spacer 10 is moved from the closed orientation to the open orientation, middle member 40 slides relative to the chassis 50 with the locking tabs 55 contacting the outer sidewalls of the middle member 40. At the open orientation as illustrated in FIG. 6, the locking tabs 55 contact the indents 47 to prevent the middle member 40 from moving in the distal direction towards the closed orientation. In the embodiment illustrated, locking tabs 55 are positioned on the chassis 50 with indents on the middle member 40. In another embodiment, the chassis 50 includes indents that receive locking tabs 55 that extend outward from the middle member 40. In one embodiment as illustrated in FIG. 6, locking tabs 55 are positioned on each sidewall 52 of the chassis 50. In another embodiment, one sidewall 52 includes a locking tab 55.

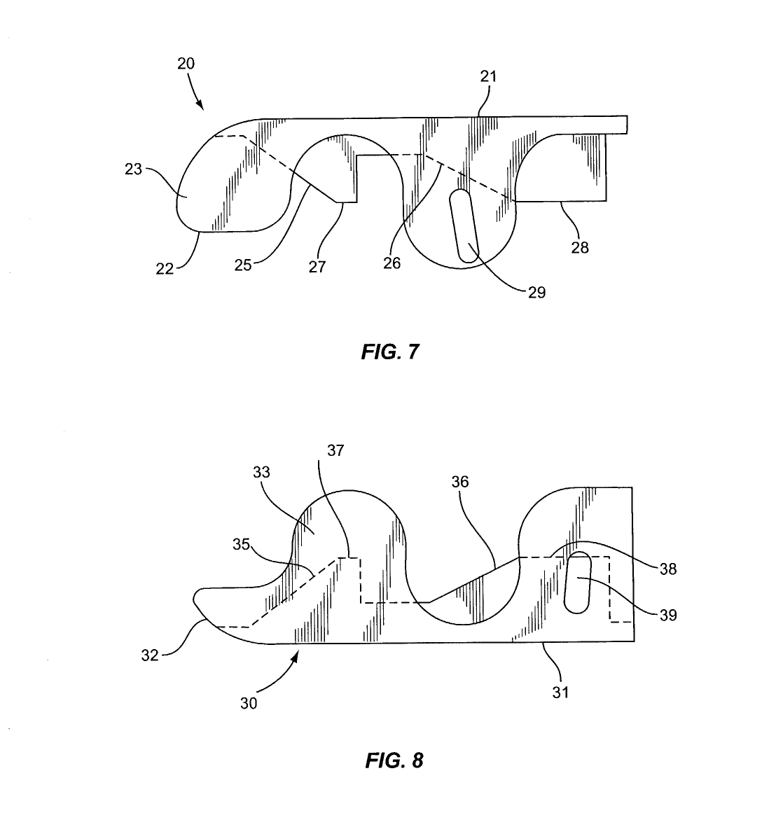

FIG. 7 illustrates one embodiment of the first member 20 which includes a contact surface 21 for contacting a vertebral member. In this embodiment, sidewalls 23 extend from the contact surface 21 to frame the exterior of the first member 20. Sidewalls 23 may have a variety of shapes and sizes to mate with the second member 30. A slot 29 is positioned on sidewalls to receive a guide mechanism as detailed below.

In the embodiment of FIG. 7, first member 20 includes a first angled edge 25 and a support surface 27 to a first wedge, and a second angled edge 26 and support surface 28 to contact a second wedge 42. Angled edges 25, 26 may have a variety of lengths, and may be positioned at a variety of angles relative to the contact surface 21. In one embodiment, the range of angles between the edges 25, 26 and contact surface 21 is between about 20° to about 40°. Support surfaces 27, 28 are positioned on a proximal side of the angled edges 25, 26. When the spacer 10 is in the open orientation, support surface 27 contacts a platform surface of the first wedge, and support surface 28 contacts a platform surface of the second wedge. Support surfaces 27, 28 are positioned at a different angle relative to the angled surfaces 25, 26. In one embodiment, support surfaces 27, 28 are substantially parallel with the contact surface 21.

FIG. 8 illustrates one embodiment of a second member 30 which compliments the first member 20. Second member 30 includes a contact surface 31 for contacting a vertebral member. In one embodiment, sidewalls 33 extend from the contact surface 31 to frame the exterior of the first member 30. Sidewalls 33 may have a variety of shapes and sizes to mate with the first member 20. A slot 39 is positioned on sidewalls to receive the guide mechanism as detailed below.

In the embodiment of FIG. 8, second member 30 includes a first angled edge 35 and adjacent support surface 37 a first wedge, and a second angled edge 36 and support surface 38 to contact a second wedge. Angled edges 35, 36 may have a variety of lengths, and may be positioned at a variety of angles relative to the contact surface 31. In one embodiment, the range of angles between edges 35, 37 and contact surface 31 is between about 20° to about 40°. Support surfaces 37, 38 are positioned at a different angle relative to the angled surfaces 35, 36. In one embodiment, support surfaces 37, 38 are substantially parallel with the contact surface 31.

A delivery device 80 is attached to the spacer 10. Delivery device 80 has an elongated shape that allows the physician to position the spacer 10 within the patient between vertebral members. In one embodiment as illustrated in FIG. 3, delivery device 80 is attached to the middle member 40. The delivery device 80 is used to position the spacer 10 between the vertebral members. In one embodiment as illustrated in FIG. 3, delivery device 80 comprises an inner member 89 movably positioned within an outer member 88. In one embodiment, delivery device 80 remains attached to the spacer 10. In another embodiment, delivery device 80 is detachable from the spacer 10. Removing the delivery device 80 may be necessary to provide additional operating space for the physician during the procedure as the delivery device 80 may interefere with other equipment, or the vision if it were left attached to the spacer 10. In this usage, the delivery device 80 may further be reattached to the spacer 10 for removal from the patient at the end of the procedure. In another usage, the delivery device 80 is removed and the spacer 10 remains permanently within the patient.

Delivery device 80 additionally provides an axial force to the middle member 40 to deploy the spacer 10 between the closed and open orientations. The axial force causes the angled surfaces of the middle member 40 to move relative to the angled surfaces of the first and second members 20, 30 to deploy the device 10. In one embodiment, the axial force is applied by linearly moving the delivery device 80. In one embodiment, the inner member 89 attached to the middle member 40 and the outer member 88 is attached to the chassis 50. The inner member 89 is axially moved relative to the outer member 88 and locked in an extended position to lock the spacer 10 in the open orientation. The inner member 89 is attached to the middle member 40 via a shearable pin that is designed to fail once member 40 is moved distally. Once the pin is sheared, the inner member 89 is withdrawn from the distal portion of outer member 88. This allows upper and lower portions of the dovetail to collapse towards axis C. This allows delivery device 80 to dislocate from chassis 50 and be removed from the spacer 10. In another embodiment, the inner member 89 moves relative to the outer member with both remaining attached to the spacer 10 such that the spacer 10 can be returned to the closed orientation and removed from the patient when the procedure is complete.

In another embodiment, delivery device 80 is attached to the spacer 10 by threads. Rotation of the delivery device 80 relative to the spacer 10 causes the spacer to dislocate from the device 80. In one embodiment, the outer member 88 includes threads that mate with threads on the spacer 10. In another embodiment, delivery device 80 and spacer 10 are equipped with a half turn locking system such that rotation of the delivery device relative to the spacer causes dislocation.

Various types of power mechanisms can be applied to the delivery device 80 to expand the spacer 10. The mechanism may be positioned adjacent to the spacer 10, or positioned distant from the spacer 10 to be outside the patient. Previously filed U.S. patent application Ser. No. 10/178960 entitled Minimally Invasive Expanding Spacer and Method, filed Jun. 25, 2002 and assigned to the same entity as the present application, discloses several different types of delivery devices and structures for deploying the spacer to the open orientation and is herein incorporated by reference in its entirety.

Guide mechanisms 70 extend through at least a portion of the spacer 10 to position the first and second members 20, 30, relative to the middle member 40 and chassis 50 and provide torsional stability to the spacer 10. In one embodiment, a first guide mechanism 70 extends through the slot 49 in the middle member 40, apertures 56 in the chassis 50, and slot 39 in the second member 30. A second guide mechanism 70 extends through the slot 49, apertures 56, and slot 29 in the first member 20.

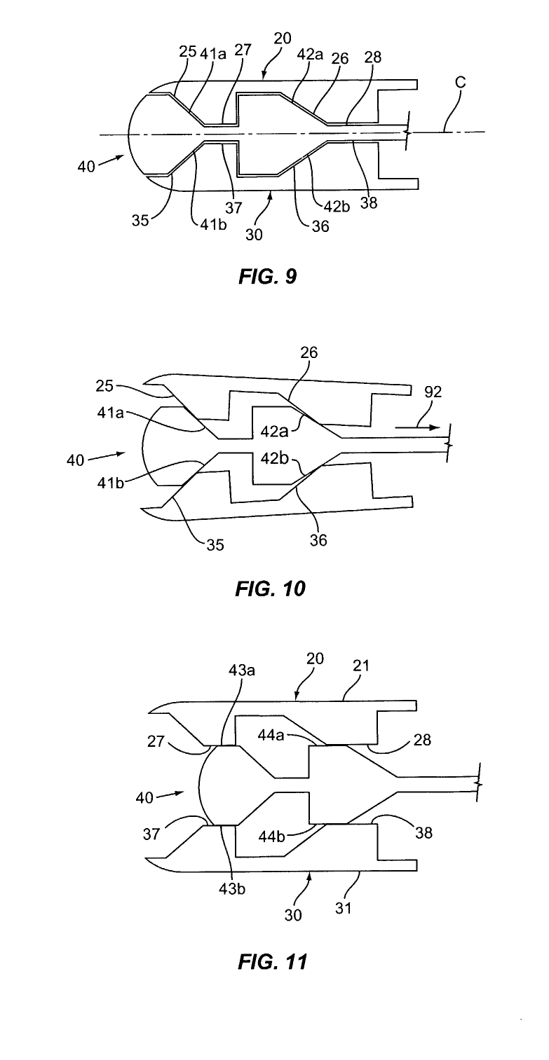

In one method of use, spacer 10 is positioned within the patient in the closed orientation as illustrated in FIG. 9. The minimal size and shape of the spacer 10 facilitates placement within the patient and between the vertebral members. In one embodiment, the angled surfaces 25, 26, 35, 36 of the first and second members 20, 30 and the ramped surfaces of the middle member 40 are spaced apart. In another embodiment, the angled surfaces are in contact with the ramped surfaces.

FIG. 10 illustrates the middle member 40 being moved in direction of arrow 92. Movement of the middle member 40 causes the angled surfaces 41a, 42a of the wedges 41, 42 to contact the angled surfaces 25, 26 of the first member 20. This causes the first member 20 to move outward away from the centerline C. Likewise, movement of the middle member 40 causes the angled surfaces 41b, 42b of the wedges 41, 42 to contact the angled surfaces 35,.36 of the second member 30 to move the second member 30 outward away from the centerline C. As the middle member 40 is pulled further inward away from the distal end of the spacer 10, the angled surfaces continue to slide relative to one another and the first and second members 20, 30 continue to move outward from the centerline C. The contact surfaces 21, 31 move outward and come into contact with the vertebral members.

FIG. 11 illustrates one embodiment at the point of full deployment. The support surfaces 27, 28 on the first member 20 contact and rest on platform surfaces 43a, 44a of the middle member 40, and support surfaces 37, 38 on the second member 30 contact and rest on platform surfaces 43b, 44b. The platform surfaces and support surfaces are angled to a lesser amount than the angled surfaces and assist to prevent the spacer 10 from moving towards the closed orientation.

In one deployment embodiment, the middle member 40 also moves relative to the chassis 50 as illustrated in FIG. 6. The middle member 40 moves in a proximal direction as the chassis 50 remains relatively stationary. During the movement, the locking tabs 55 on the chassis 50 slide along sidewalls of the middle member 40. At the open orientation, the locking tabs 55 on the chassis 50 move inward and contact the indents 47 of the middle member 40. This placement prevents the middle member 40 from moving in a distal direction which would cause the spacer 10 to close.

The slope or sizes of the wedges 41, 42 and the angled surfaces of the first and second members 20, 30 may vary to change the shape of the spacer 10 in the open orientation. In one embodiment illustrated in FIG. 2, contact surfaces 21 and 31 are oblique with the distal end of the spacer 10 having a larger height than the proximal end. This shaping is caused by either the distal wedge 41b being larger than the proximal wedge 42, the distal angled edges 25, 35 of the first and second members 20, 30 being larger than the proximal angled edges 27, 37, or a combination of both. In one embodiment, the spacer 10 in the open orientation is shaped to conform to the curvature of the spine. In another embodiment as illustrated in FIG. 11, the contact surfaces 21, 31 are substantially parallel at the open orientation.

In one embodiment, the spacer 10 expands in a single direction as one of the first and second members 20, 30 moves outward from the centerline C during the opening process. By way of example, first member 20 may have angled surfaces that ride along angled surfaces within a middle member 40 to deploy to the open position. Second member 30 does not include angle surfaces, or middle member 40 does not include angled edges 41b, 42b, or both, and thus does not deploy. In another embodiment, one of the first or second members 20, 30 deploys to a lesser extent than the other member. By way of example, first member 20 may deploy a distance X from the centerline C between the closed and open orientations and second member 30 deploys a distance less than X.

In one embodiment, the spacer 10 includes two members that each have angled surfaces and there is no middle member. The angled surfaces of the first member contacts the angled surfaces of the second member during the deployment.

In one embodiment, a single angled surface provides movement of the spacer. In this embodiment, the middle member 40 includes a single wedge and first and second members 20, 30 each include a single angled surface that contacts the wedge. Likewise, in an embodiment that does not include a middle member 40, the two members each include a single angled surface that contacts each other during the opening process.

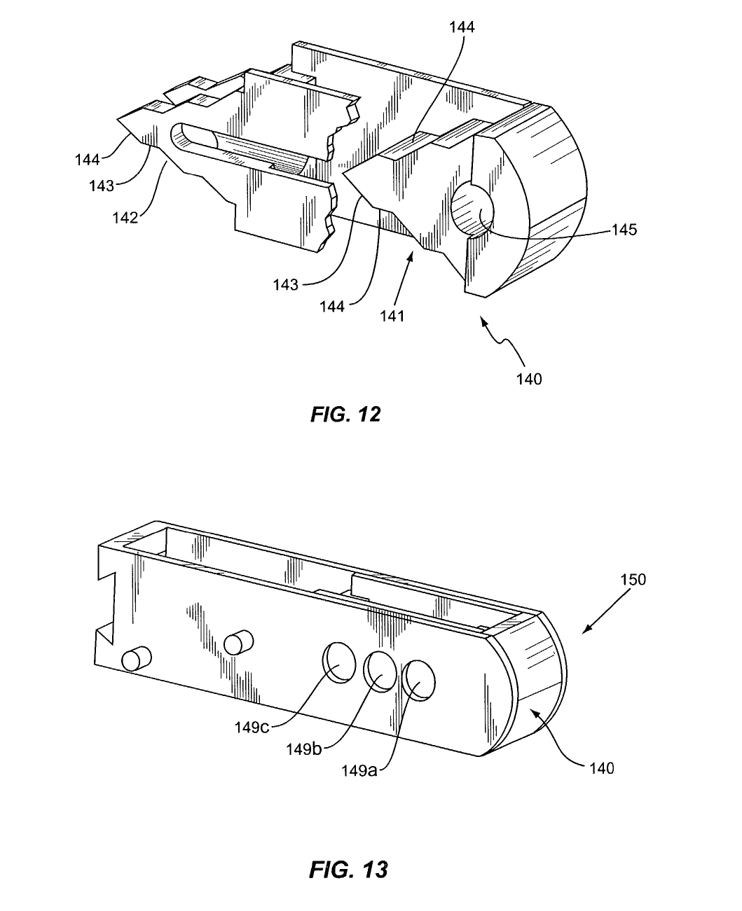

Another embodiment of a middle member 140 is illustrated in FIG. 12. Middle member 140 comprises one or more stepped wedges 141, 142 that each include angled surfaces 143, and step surfaces 144. The different placements of the step surfaces 144 along the wedge provide for opening the spacer to a variety of different heights. Support surfaces on the first and second members 20, 30 rest on the contact surfaces for support at the different placements. A variety of step surfaces 144 may be positioned on the wedges 141, 142. In the embodiment of FIG. 12, the spacer can be positioned between a closed orientation, first orientation on the first step, second orientation on the second step, and fully deployed orientation on the third step. The spacer may include both wedges 141, 142 having a stepped configuration, or one of the wedges having a stepped configuration.

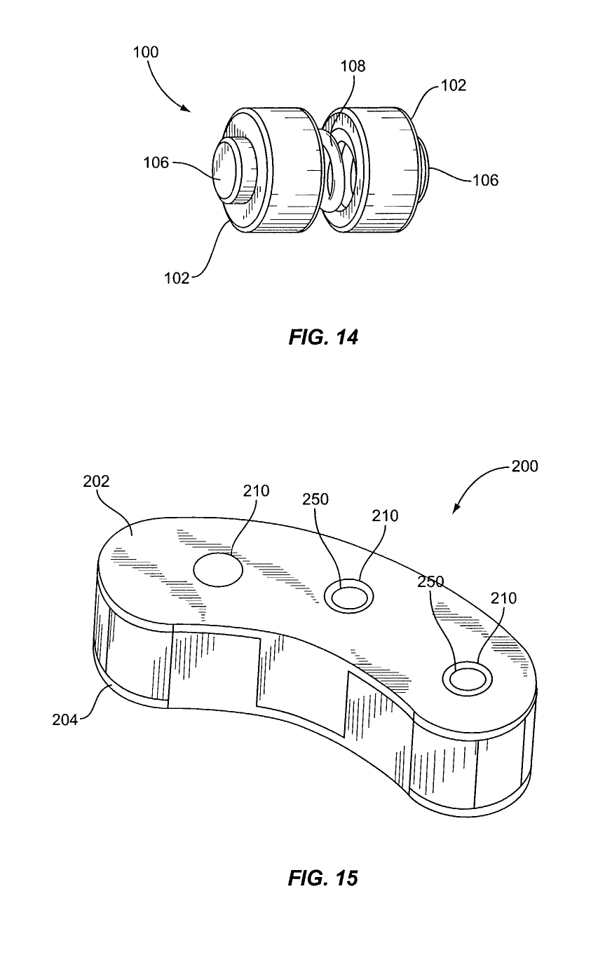

FIG. 13 illustrates an alternative embodiment of a chassis 150. Sidewalls of the chassis include a plurality of apertures 149 (149a, 149b, 149c). The placement of the apertures 149 coincide with the placement of the support surfaces on the stepped wedges. The apertures 149 provide a position for locking the middle member 140 relative to the chassis 150.

In one embodiment, a locking member 100 as illustrated in FIG. 14 is positioned within an opening 145 of the middle member 140 and mates with the apertures 149 for locking the middle member 140 relative to the chassis 150. Locking member 100 includes a pair of caps 102 biased apart by a biasing member 108. Each cap 102 includes an extension 106 sized to fit within the apertures. As the middle member 140 moves relative to the chassis 150 along the stepped wedge or wedges, the locking member 100 extends into one of the apertures 149. By way of example, as the middle member 140 moves such that the platform surfaces of the first and second members contact the first step surface, the extensions extend through first aperture 149a. As the middle member moves to the second step, extension extends through second aperture 149b. At full deployment, extensions 106 are positioned within third aperture 149c. Chassis 150 may include an aperture 149 for each of the steps within the wedge or wedges, or may have an aperture 149 for only a limited number of steps.

In another embodiment, locking member 100 is used on a middle member having ramped wedges (i.e., without steps). At various positions of deployment, openings in the chassis may be positioned to receive the extensions of the caps to lock the spacer 10.

FIG. 15 illustrates another embodiment of a spacer 200 having a different orientation. This embodiment has a generally kidney-shape with a curved body and opposite rounded ends. Spacer 200 may be positioned at a variety of heights with first member 202 spaced apart from second member 204 at a variety of distances.

The term vertebral member is used generally to describe the vertebral geometry comprising the vertebral body, pedicles, lamina, and processes. The spacer 10 may be sized and shaped, and have adequate strength requirements to be used within the different regions of the vertebra including the cervical, thoracic, and lumbar regions. In one embodiment, spacer 10 is positioned within the disc space between adjacent vertebras. Contact surfaces 21, 31 contact the end plates of the vertebra to space the vertebra as necessary. In one embodiment, the spacer 10 is inserted posteriorly in the patient. In another embodiment, the spacer 10 is inserted from an anteriorly into the patient. In another embodiment, the spacer is inserted laterally into the patient.

In one embodiment, contact surfaces 21, 31 are porous to allow bone ingrowth into the spacer 10. In one embodiment, one or both contact surfaces 21, 31 include one or more apertures. One example is illustrated in FIG. 15 with apertures 210 positioned about the first member 202. Apertures may also be positioned within the second member 204. Bone growth material 250 is positioned within the apertures 210 to accommodate bone growth through the entire implant. In one embodiment, apertures within the first member 202 coincide with apertures in the second member 204. The bone growth material may be include a sponge, matrix, and/or other carrier impregnated with a protein such as bone morphogenic protein (BMP), LIM mineralization protein (LMP), etc.

The spacer of the present invention may have a variety of shapes and sizes. In one embodiment, the spacer in a closed orientation has a length of about 21 mm, a width of about 7 mm, and a height of about 7.5 mm. In one embodiment, first and second contact surfaces 21, 31 have dimensions of about 17 mm by 7 mm. In one embodiment, the deployed spacer has a lordotic angle of about 14.2°, a posterior height of about 9.9 mm and an anterior height of about 14.2 mm. In another embodiment, the size of the spacer in the closed orientation is about 7.5 mm in height, about 7 mm in width, and about 20 mm in length. The size of the spacer in the open orientation is about 10.25 mm in posterior height with an anterior height of about 14.25 mm.

The present invention may be carried out in other specific ways than those herein set forth without departing from the scope and essential characteristics of the invention. In one embodiment, spacer 10 and delivery device 80 are constructed of stainless steel. In one embodiment the biasing member 108 is a coil spring. In one embodiment, when the spacer 10 is in the open orientation the angled surfaces of the members are in contact. The present embodiments are, therefore, to be considered in all respects as illustrative and not restrictive, and all changes coming within the meaning and equivalency range of the appended claims are intended to be embraced therein.

What is claimed is:

1. A spacer to position vertebral members comprising:
a first body comprising a first angled surface that increases distally along a length of the spacer;
a second body adjacent to the first body and comprising a second angled surface that increases proximally along a length of the spacer;
the first body being movable relative to the second body and positionable between a first orientation having a first height, and a second orientation having a second height with the first angled surface and the second angled surface being in contact, the second height being greater than the first height.

2. The spacer of claim 1, wherein the first angled surface and the second angled surface are spaced apart in the first orientation.

3. The spacer of claim 1, wherein the first body comprises a first contact surface positioned opposite from the first angled surface, and the second body comprises a second contact surface positioned opposite from the second angled surface.

4. The spacer of claim 3, wherein the first and second contact surfaces are substantially parallel in the first orientation.

5. The spacer of claim 3, wherein the first and second contact surfaces are oblique in the second orientation.

6. The spacer of claim 1, wherein the first angled surface has a different slope than the second angled surface.

7. The spacer of claim 1, further comprising a third angled surface on the first body that contacts a fourth angled surface on the second body when moving the spacer from the first orientation to the second orientation.

8. A spacer for positioning between vertebral members comprising:
a middle member having a wedge with a first surface and a second surface forming a vertex that is positioned proximally of a base;
a first member positioned on a first side of the middle member and comprising a first contact surface and a first angled surface; and
a second member positioned on a second side of the middle member and comprising a second contact surface and a second angled surface;
the device being positionable between a first orientation with the first angled surface positioned from the first surface and the second angled surface positioned from the second surface, and a second orientation with the first angled surface contacting the first surface and the second angled surface contacting the second surface with the first contact surface and the second contact surface positioned apart a distance greater than in the first orientation.

9. The spacer of claim 8, wherein the vertex of the middle member is positioned on a centerline of the spacer.

10. The spacer of claim 8, wherein the first contact surface and the second contact surface are substantially parallel in the first orientation and oblique in the second orientation.

11. The spacer of claim 8, further comprising a chassis with locking tabs that mate with the middle member in the second orientation to prevent the spacer from moving from the second orientation towards the first orientation.

12. The spacer of claim 8, wherein the first angled surface has a different slope than the second angled surface.

13. The spacer of claim 8, wherein the first surface and the second surface each have a stepped configuration.

14. The spacer of claim 8, further comprising a locking member positioned within the middle member that locks with a chassis, the locking member comprising a spring-biased cap that extends outward from the middle member.

15. A spacer for positioning between vertebral members comprising:
a middle member having a first wedge and a second wedge each angled in a first direction and positioned a predetermined distance apart;
a first member comprising a first contact surface, and a first angled surface and a second angled surface positioned the predetermined distance apart, the first angled surface and the second angled surface angled in a second direction different from the first direction; and
a second member comprising a second contact surface and a third angled surface and a fourth angled surface positioned the predetermined distance apart, the second member being positioned adjacent to the middle member and opposite from the first member, the third angled surface and the fourth angled surface angled in the second direction.

16. The spacer of claim 15, comprising a chassis having a locking tab that contacts the middle member to lock the spacer in the open orientation.

17. The spacer of claim 16, wherein the middle member comprises an indent positioned along a sidewall to receive the locking tab.

18. The spacer of claim 15, further comprising a locking member having a pair of extensions that extend outward to mate with a chassis positioned around the middle member.

19. The spacer of claim 18, wherein the chassis comprises a plurality of apertures spaced to receive the locking member to lock the spacer in a variety of open orientations.

20. The spacer of claim 15, further comprising an elongated delivery device attached to a proximal end of the spacer.

21. The spacer of claim 20, further comprising detachment means for separating the delivery device from the spacer.

22. The spacer of claim 15, wherein the first wedge has a height different than the second wedge.

23. The spacer of claim 15, wherein one of the first and second angled surfaces and one of the third and fourth angled surfaces comprise stepped surfaces.

24. The spacer of claim 15, further comprising guiding mechanisms that extend through the middle member, first member, and second member.

25. A spacer for positioning between vertebral members comprising:
a first body comprising a first angled surface and a platform surface positioned at a first angled surface end; and
a second body comprising a second angled surface and a support surface positioned at a second angled surface end, the first angled surface positioned in a different direction from the second angled surface;
the first body being movable relative to the second body between a first orientation with the platform surface apart from the support surface and a second orientation with the platform surface contacting the support surface, a height of the spacer being greater in the second orientation than in the first orientation.

26. The spacer of claim 25, wherein the first angled surface is angled in a first direction relative to a distal end of the spacer, and the second angled surface is angled in a second direction relative to the distal end of the spacer, the first direction being different than the second direction.

27. A spacer for positioning between vertebral members comprising:
a wedge having a first platform surface and a second platform surface;
a first member comprising a first angled surface having a first support surface positioned at an end of the first angled surface;
a second member comprising a second angled surface having a second support surface positioned at an end of the second angled surface;
the spacer being positionable between a first orientation with the first support surface apart from the first platform surface and the second support surface apart from the second platform surface, and a second orientation with the first support surface contacting the first platform surface and the second support surface contacting the second platform surface, a spacer height being greater in the second orientation than in the first orientation;
the first angled surface and second angled surface being in the same direction.

28. A spacer for positioning vertebral members comprising:
a middle member having an angled wedge with a first step and a second step;
a first member having a first angled surface and a first support surface;
a second member having a second angled surface and a second support surface;
a chassis positioned around the middle member and having a first aperture and a second aperture spaced a distance apart;
a locking member positioned within the middle member;
the middle member being movable relative to the first and second members between a first position with the first support surface and the second support surface positioned on the first step and the locking member positioned within the first aperture, and a second position with the first support surface and the second support surface positioned on the second step and the locking member positioned within the second aperture, the spacer having a height that is greater in the second position than in the first position.

29. The spacer of claim 28, wherein the first angled surface and second angled surface extend in a first direction, and the angled wedge extends in a second direction different than said first direction.

30. The spacer of claim 28, wherein the locking member comprises a pair of caps separated by a biasing member.

31. A method of spacing a first vertebral member from a second vertebral member comprising the steps of:
inserting a spacer between the vertebral members;
increasing a height of the spacer by sliding an angled surface of a first member against an angled surface of a second member and separating a first contact surface from a second contact surface; and
contacting the first vertebral member with the first contact surface and contacting the second vertebral member with the second contact surface.

32. The method of claim 31, further comprising locking the spacer and preventing the first contact surface from separating from the first vertebral member and the second contact surface from separating from the second vertebral member.

33. The method of claim 31, wherein the step of inserting a spacer between the vertebral members comprises manipulating a delivery device and positioning the spacer between the vertebral members.

34. A method of spacing a first vertebral member from a second vertebral member comprising the steps of:
moving a first member away from a centerline of a spacer by sliding a first angled surface of the first member against a first inclined surface of a middle member;
moving a second member away from a centerline of the spacer by sliding a second angled surface of the second member against a second inclined surface of the middle member; and
contacting the first member with the first vertebral member and contacting the second member with the second vertebral member.

35. The method of claim 34, further comprising placing bone growth material within the spacer.

36. The method of claim 34, further comprising locking the spacer in an open orientation by mating a locking mechanism with the middle member, with the first contact surface against the first vertebral member and the second contact surface against the second vertebral member.

37. The method of claim 34, further comprising moving a flat first support surface of the first member against a flat first platform surface of the middle member, and moving a flat second support surface of the second member against a flat second platform surface of the middle member.

38. The method of claim 34, further comprising positioning the first member and the second member at oblique angles relative to the centerline.

39. A method of spacing vertebral members comprising the steps of:
placing a spacer in a first orientation between the vertebral members, the spacer having a first height;
moving a middle member in a distal direction relative to a first member and a second member; and
increasing the spacer to a second height greater than the first height by sliding an angled wedge of the middle member against a first angled edge of a first member and a second angled edge of a second member.

40. The method of claim 39, further comprising placing a first support surface of the first member against a first platform surface of the middle member, and placing a second support surface of the second member against a second platform surface of the middle member.

41. The method of claim 39, further comprising locking the middle member at the second height by positioning locking tabs in a chassis against the middle member.

42. The method of claim 39, further comprising locking the middle member at the second height by positioning a locking member within an aperture within a chassis.
